(19) United States
(12) Patent Application Publication
(10) Pub. No.: US 2009/0308772 A1
(43) Pub. Date: Dec. 17, 2009

(54) MOISTURE-TIGHT PRIMARY PACKAGING FOR A DRY POWDER INHALER

(76) Inventor: William S. Abrams, Auburn, AL (US)

Correspondence Address: GREENBERG TRAURIG, LLP, MET LIFE BUILDING, 200 PARK AVENUE, NEW YORK, NY, 10166, US

(21) Appl. No.: 12/282,419

(22) PCT Filed: Mar. 19, 2007
(86) PCT No.: PCT/US07/64296

§ 371 Date: Jan. 30, 2009

Related U.S. Application Data
(60) Provisional application No. 60/783,270, filed on Mar. 17, 2006.

(51) Int. Cl. B65D 81/26; A61M 15/00

(52) U.S. Cl. 206/438; 128/203.15

(57) ABSTRACT

In one embodiment, the present invention relates to a primary package that houses a dry powder inhaler comprising: a) the dry powder inhaler, wherein the dry powder inhaler comprises a mouthpiece, body and actuator; b) the primary package, when in a closed position, completely houses the dry powder inhaler within an inner portion of the package, wherein: i) the primary package comprises a resealable mechanism, ii) the dry powder inhaler is mechanically secured to the primary package so that, when to primary package is in an open position, the mouthpiece and actuator of the dry powder inhaler is accessible while, simultaneously, the dry powder inhaler is secured within the primary package, and iii) the primary package has a sealant mechanism so that, when the primary package is in the closed position, a moisture-tight seal is formed.

### BACKGROUND

Dry Powder Inhalers (DPI) are medical devices used to deliver Active Pharmaceutical Ingredients (APIs) into the lungs. Based on the design, the DPI could deliver single or multiple APIs in one inhalation dosage. DPIs are typically packaged in foil bags to ensure physical and chemical stability of the powders and APIs during shelf life. However, when the user removes the inhaler from the foil bag, there is little or no protection of the product during use life. Moreover, as new APIs are discovered and commercialized, it is possible that additional protection from moisture may be required to ensure physical and/or chemical stability during product Use Life.

### SUMMARY OF THE INVENTION

In one embodiment, the present invention relates to a primary package that houses a dry powder inhaler comprising: a) the dry powder inhaler, wherein the dry powder inhaler comprises a mouthpiece, body and actuator; b) the primary package, when in a closed position, completely houses the dry powder inhaler within an inner portion of the package, wherein: i) the primary package comprises a resealable mechanism, ii) the dry powder inhaler is mechanically secured to the primary package so that, when to primary package is in an open position, the mouthpiece and actuator of the dry powder inhaler is accessible while, simultaneously, the dry powder inhaler is secured within the primary package, iii) the primary package has a sealant mechanism so that, when the primary package is in the closed position, a moisture-tight seal is formed. In another embodiment, the primary package includes a desiccant composition within the inner portion of the package.

In another embodiment, the present invention relates to a primary package further comprising an integral cap and a container portion, wherein a hinge is integrally connected to the cap portion and the container portion. In another embodiment, the present invention relates to a primary package further comprising a cap and a container portion, wherein the individually molded portions are snap-fit or welded together. In another embodiment, the present invention relates to a primary package wherein the mechanical attachment is accomplished using a snap fit or sonic welding. In another embodiment, the present invention relates to a primary package wherein the mechanical attachment is a ratchet mechanism so that, when the primary package is opened, the dry powder inhaler is lifted out of the primary package. In another embodiment, the present invention relates to a primary package wherein the desiccant composition is a sachet or canister containing a desiccant. In another embodiment, the present invention relates to a primary package wherein the desiccant composition is a desiccant entrained in a polymer and wherein at least a portion of inner walls of the primary package is comprised of the desiccant composition. In another embodiment, the present invention relates to a primary package wherein the primary package is an injection-molded part. In another embodiment, the present invention relates to a primary package further comprising a thermoplastic elastomer in at least a portion of the sealing area of the primary package.

### BRIEF DESCRIPTION OF THE DRAWINGS

A more complete appreciation of the present invention and many of the attendant advantages thereof will be readily understood by reference to the following description was considered in connection with the accompanying drawings in which:

FIG. 1 is a cut-away view of a side elevation of one embodiment of a DPI;

FIG. 2A is a cut-away view of a side elevation of a DPI packaged in one embodiment of the present invention;

FIGS. 2B and 2C are an illustrative view of one embodiment of the present invention as may be employed by a user;

FIG. 3A is a cut-away view of a side elevation of a DPI packaged in one embodiment of the present invention;

FIG. 3B is an illustrative view of one embodiment of the present invention as may be employed by a user;

FIG. 4A is a side elevation view of a DPI packaged in one embodiment of the present invention;

FIG. 4B is an illustrative view of one embodiment of the present invention as may be employed by a user;

FIG. 5A is a side elevation view of a DPI packaged in one embodiment of the present invention; and

FIG. 5B is an illustrative view of one embodiment of the present invention as may be employed by a user.

Among those benefits and improvements that have been disclosed, other objects and advantages of this invention will become apparent from the following description taken in conjunction with the accompanying drawings. The drawings constitute a part of this specification and include exemplary embodiments of the present invention and illustrate various embodiments and features thereof.

### DETAILED EMBODIMENTS OF THE INVENTION

Detailed embodiments of the present invention are disclosed herein; however, it is to be understood that the disclosed embodiments are merely illustrative of the invention that may be embodied in various forms. In addition, each of the examples given in connection with the various embodiments of the invention are intended to be illustrative, and not restrictive, Further, the figures are not necessarily to scale, some features may be exaggerated to show details of particular components. Therefore, specific structural and functional details disclosed herein are not to be interpreted as limiting, but merely as a representative basis for teaching one skilled in the art to variously employ the present invention.

In one embodiment, the present invention relates to a primary package that permanently houses the DPI to ensure product protection during Use Life. For example, the primary package is a flip-top container, that when opened by a user, provides access to the Dry Powdered Inhaler. As such, the user is able to access the mouthpiece and actuator of the DPI without having to remove completely the DPI from the primary package.

In another embodiment, the primary package is mechanically attached to the DPI. In one specific embodiment, the mechanical attachment is accomplished using a snap fit or sonic welding. In another embodiment, the mechanical attachment is accomplished using a ratchet mechanism to attach the components so that the DPI can be positioned (tilted) within the primary package. When the primary packaged is closed, a moisture-tight seal is created that substantially prevents moisture from entering the DPI.

The result of the present invention is, for example, that the user is not required to remove completely the DPI from the primary package. By not requiring the user to remove completely the DPI from the primary package, the risk that the primary package might be discarded is reduced. In addition, by not requiring the user to remove completely the DPI from the primary package, the risk is that the user simply forgets to return the DPI for a period of time where it would be exposed to the ambient environment is reduced. As such, the present invention allows the user to access the DPI while it remains in the primary package and thus, can be reclosed.

In another embodiment, the primary package provides adequate protection during “Use Life” and thus, reduces the need for additional barrier properties in the secondary packaging. In yet another embodiment, the primary package of the present invention may include a desiccant composition within the package to absorb moisture that may enter the primary package during shelf life and/or during use life.

Suitable desiccant compositions include, but are not limited to, traditional sachet or canister. In another embodiment, the desiccant composition may be entrained in a polymer and at least a portion of the inner walls (e.g. the sidewalls) of the primary package is composed of the desiccant composition. In one example, the sleeve, composed of plastic composition, may be composed of an active agent (e.g. desiccant) mixed with a plastic (e.g. desiccant entrained plastic). The desiccant entrained plastic sleeve reduces or eliminates the need for a separate desiccant canister or sachet. One example is a three component composition is the compositions and methods disclosed in one or more of the following U.S. Pat. Nos. 5,911,937, 6,214,255, 6,130,263, 6,080,350 and 6,174,952, 6,124,006, and 6,221,446. These references are incorporated herein by reference. Another example is a two component mixture of active agent and plastic.

In one embodiment, the primary package is made using an injection-molded process. The primary package may be made from thermoplastic resins. Suitable materials for the primary package include, but are not limited to, polypropylene, high-density polyethylene, polyethylene and/or combinations thereof. In another embodiment, a thermoplastic elastomer may be used in the scaling area to provide the moisture-tight seal. Suitable elastomeric materials include, but are not limited to, EPDM, Santoprene, and combinations thereof.

Suitable methods for making the present invention include, but are not limited to, the following method disclosed in the following patents (hereby incorporated by reference herein): the flip-top lid creates a substantially moisture-tight resealable closure by closing the lid during the molding process. Examples (although not limiting) of flip-top lids designs are described in U.S. Patent Nos. Re 37,676, U.S. Pat. Nos. 4,812,116, and 4,783,056, all of which are incorporated herein by reference.

In one embodiment, the desiccant-entrained plastic contains a desiccant such as silica gel or molecular sieve as the desiccant. Depending on the application, such as the application intended by the end user, molecular sieve or silica gel desiccant can be provided in the sleeve. For example, molecular sieve can be used for applications that require a low RH (e.g. less than <10% RH) maintained over the shelf life. In another example, silica gel can maintain a RH of 10-30% over a two-year shelf life.

In yet another embodiment, the containers of the present invention are formed of plastic materials, by injection molding or other suitable molding techniques. For example, the containers may be molded of polypropylene. In one embodiment, the containers can be formed as a single closed unit, with the hinge joining the cap portion to the container portion. Alternatively, the pieces can be molded separately, the container portion can be loaded with the item to be retained in the container, and then the container and cap portion can be snap-fit together and/or welded, in order to provide moisture-tightness. In yet another embodiment, the cap and container portions can be formed as separate parts, loaded with the desired item, the container and cap can be fit together, and the parts joined, e.g., by welding (such as by sonic welding or by thermal welding). In yet another embodiment, the cap and container can be formed in a single piece (for example, joined at the hinge), yet open, as in a clamshell arrangement. The assembly can be filled with contents, closed shut, and then joined, e.g., welded to form a seat as described above.

In another embodiment, the present invention relates to a container that is moisture-tight and resealable mechanism. The term “resealable” means that the container can be opened/reopened and closed/reclosed a numerous amount of times (e.g. more than 5 times) and still retain its moisture-tight properties. The term “moisture tight” means the moisture ingress of the container was less than about 1500 micrograms/day of water, determined by the following test method: (a) place approximately one gram of molecular sieve in the container and record the weight; (b) close the resealable mechanism; (c) place the seated container in an environmental chamber at conditions of 80% relative humidity and 72 F (22 C); (c) after one day, weigh the container containing the molecular sieve; (d) after approximately two weeks, weigh the container (e) subtract the first day sample from the value obtained and divide by the number of days to calculate the moisture ingress of the container in units of micrograms of water.

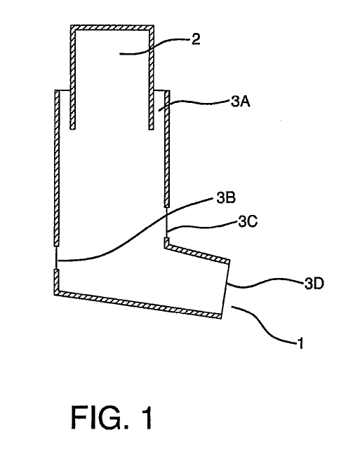

The following discussion of the Figures are to be used as illustrative examples and not meant to limit the present invention to the examples illustrated in the Figures. FIG. 1 is a cut-away view of a side elevation of one embodiment of a DPI. The conventional DPI shown has mouthpiece 1 and manual piston mechanism 2. Items 3A-3D illustrate some of the potential locations for moisture ingress into the DPI.

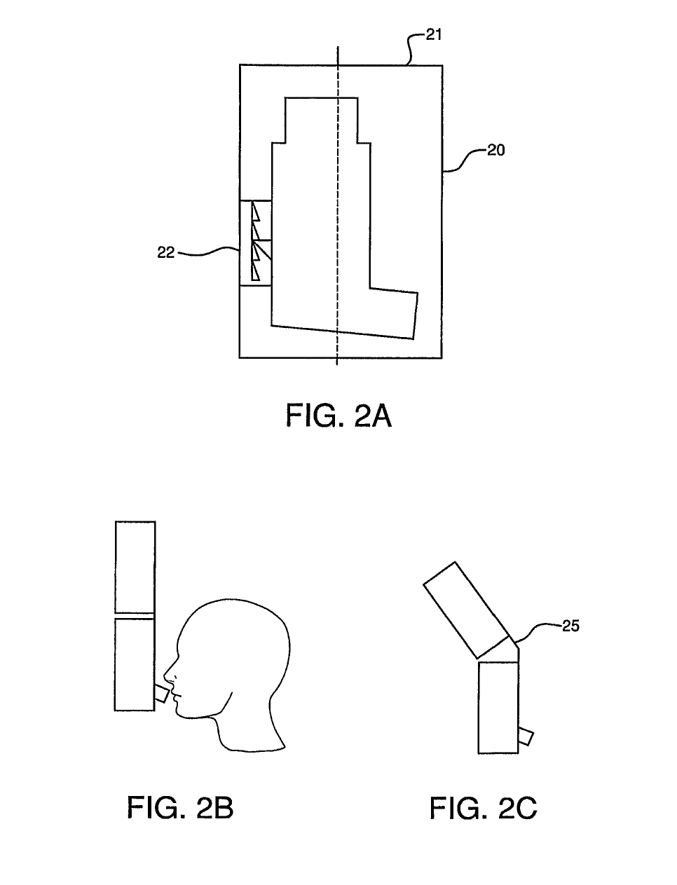

FIG. 2A is a cut-away view of a side elevation of a DPI packaged in one embodiment of the present invention. FIG. 2A is an embodiment of a rectangular-shaped, resealable primary package that protects the Inhaled Medication during the Use Life. Primary Package 20 encloses DPI 21. The mechanical attachment of the DPI is accomplished using a ratchet mechanism 22 to attach the components so that the DPI can be positioned (tilted) within the primary package 20. When the primary packaged is closed, a moisture-tight seal is created that substantially prevents moisture from entering the DPI. FIGS. 2B and 2C are an illustrative view of one embodiment of the present invention as may be employed by a user. FIG. 2C shows that, with the elongated hinge 25, the top half of primary package 20 can be folded back so that the DPI is fully accessible for use.

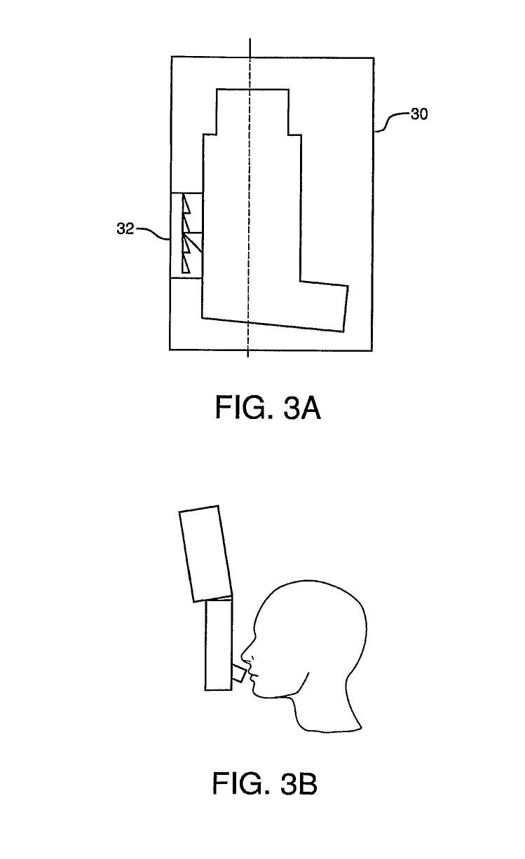

FIG. 3A is a cut-away view of a side elevation of a DPI packaged in one embodiment of the present invention. Primary Package 30 encloses DPI 31. The mechanical attachment of the DPI is accomplished using a ratchet mechanism 32 to attach the components so that the DPI can be positioned (pulled out) within the primary package 30. When the primary packaged is closed, a moisture-tight seal is created that substantially prevents moisture from entering the DPI. Since primary package 30 comprises an asymetric package, this creates two unequal sides within primary package 30. FIG. 2B is an illustrative view of one embodiment of the present invention as may be employed by a user. FIG. 3B shows that, with the creation of two unequal sides, the DPI is pulled down from the primary package using ratchet mechanism 32 to assist in the movement and to secure DPI 31 with primary package 30.

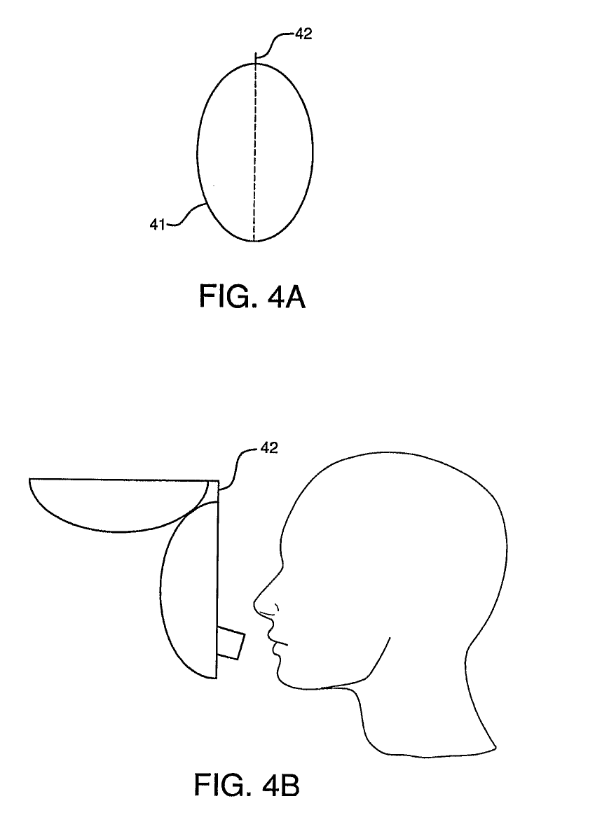

FIG. 4A is a side elevation view of a DPI packaged in another embodiment of the present invention. In this example, primary package 41 is a “clam-shape” with primary package having hinge 42. FIG. 4B is an illustrative view of package 41, in the open position, as may be employed by a user. As shown, hinge 42 is positioned with primary package having an approximate centerline divide so that, when primary package 41 is opened, primary package 41 splits into 2 approximately equal portions so as to provide access to the DPI.

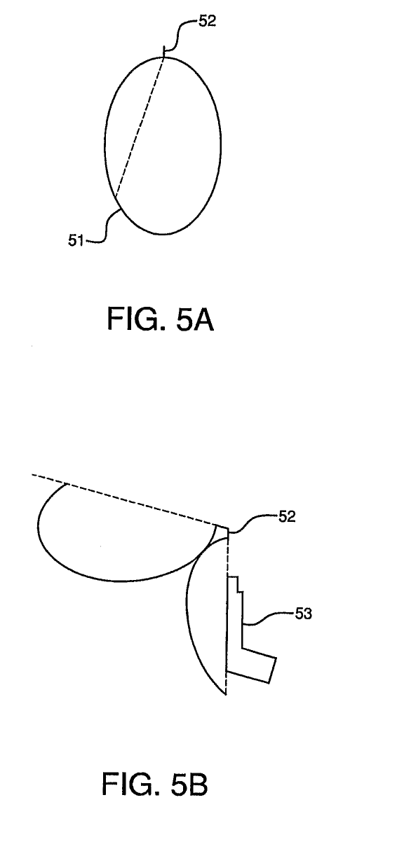

FIG. 5A is a side elevation view of a DPI packaged in another embodiment of the present invention. In this example, primary package 51 is a “clam-shape” with primary package having hinge 52. FIG. 5B is an illustrative view of package 51, in the open position, as may be employed by a user. As shown, hinge 52 is positioned with primary package having an approximate quarter-line divide so that, when primary package 51 is opened, primary package 51 splits into 2 portions—approximately 34 and approximately ¼ portions—so as to provide access to DPI 53.

While a number of embodiments of the present invention have been described, it is understood that these embodiments are illustrative only, and not restrictive, and that many modifications may become apparent to those of ordinary skill in the art. For example, a tamper-proof, tamper-resistant and/or child-resistant seal may be incorporated into the seal of the primary package.

What is claimed is:

1) A primary package that houses a dry powder inhaler comprising:
a) the dry powder inhaler, wherein the dry powder inhaler comprises a mouthpiece, body and actuator;
b) the primary package, when in a closed position, completely houses the dry powder inhaler within an inner portion of the package, wherein:
i) the primary package comprises a resealable mechanism,
ii) the dry powder inhaler is mechanically secured to the primary package so that, when to primary package is in an open position, the mouthpiece and actuator of the dry powder inhaler is accessible while, simultaneously, the dry powder inhaler is secured within the primary package, and
iii) the primary package has a sealant mechanism so that, when the primary package is in the closed position, a moisture-tight seal is formed.

2) The primary package of claim 1 wherein the primary package includes a desiccant composition within the inner portion of the package.

3) The primary package of claim 1 further comprising a cap and a container portion, wherein the individually molded portion are be snap-fit or welded together.

4) The primary package of claim 1 wherein the mechanical attachment is accomplished using a snap fit or sonic welding.

5) The primary package of claim 1 wherein the mechanical attachment is a ratchet mechanism so that, when the primary package is opened, the dry powder inhaler is lifted out of the primary package.

6) The primary package of claim 21 wherein the desiccant compositions is a sachet or canister containing a desiccant.

7) The primary package of claim 2 wherein the desiccant compositions is a desiccant entrained in a polymer and wherein the desiccant composition lines at least a portion of inner walls of the primary package.

8) The primary package of claim 1 wherein the primary package is an injection-molded part.

9) The primary package of claim 1 further comprising a thermoplastic elastomer in at least a portion of a sealing area of the primary package.
